(19) United States
(12) Patent Application Publication
(10) Pub. No.: US 2014/0363391 A1
(43) Pub. Date: Dec. 11, 2014

(54) SYSTEM AND METHOD FOR ADMINISTERING POLOXAMERS AND ANESTHETICS WHILE PERFORMING CPR AND MINIMIZING REPERFUSION INJURY

(71) Applicants: Demetris Yannopoulos, St. Paul, MN (US); Keith G. Lurie, Minneapolis, MN (US)

(72) Inventors: Demetris Yannopoulos, St. Paul, MN (US); Keith G. Lurie, Minneapolis, MN (US)

(21) Appl. No.: 14/297,976

(22) Filed: Jun. 6, 2014

Related U.S. Application Data
(60) Provisional application No. 61/832,062, filed on Jun. 6, 2013.

(51) Int. Cl. A61K 31/77; A61H 31/00; A61K 45/06

(52) U.S. Cl. CPC A61K 31/77; A61K 45/06; A61H 31/00
USPC 424/78.3; 601/41

(57) ABSTRACT

According to an embodiment, a system for delivering a synthetic surfactant and an anesthetic to an individual to reduce reperfusion injury includes an anesthetic delivery device and a device for administering the synthetic surfactant intravenously or intraosseously. The anesthetic delivery device includes a patient connection mechanism for coupling with an airway of the individual, an intrathoracic pressure regulation (IPR) mechanism that involves changing the pressure in the airway that is coupled with the patient connection mechanism, and an anesthetic delivery mechanism for receiving the anesthetic and for delivering the anesthetic to the individual via the patient connection mechanism.

### CROSS-REFERENCES TO RELATED APPLICATIONS

This application claims priority to U.S. Patent Application No. 61/832,062 filed Jun. 6, 2013, entitled “System and Method for Administering Poloxamers and Anesthetics While Performing CPR and Minimizing Reperfusion Injury,” the entire disclosure of which is hereby incorporated by reference, for all purposes, as if fully set forth herein. The application is also related to the following U.S. patents applications, each of which are incorporated by reference herein: Ser. Nos. 07/686,542; 08/058,195; 08/226,431; 07/977,498; 08/149,204; 08/403,009; 08/747,371; 08/950,702; 09/019,843; 09/095,916; 09/168,049; 09/197,286; 09/315,396; 09/386,868; 09/614,064; 09/532,601; 09/533,880; 09/564,889; 09/546,252; 09/704,231; 09/854,404; 09/854,238; 09/966,945; 09/967,029; 10/119,203; 10/158,528; 10/251,080; 10/224,263; 10/255,319; 10/401,493; 10/410,229; 10/396,007; 10/426,161; 10/460,558; 10/660,366; 10/660,462; 10/765,318; 10/796,875; 10/920,678; 11/034,996; 11/051,345; 11/127,993; 11/735,924; 11/679,693; 11/690,065; 11/735,320; 60/912,891; 60/917,602; 60/944,735; 60/947,346; 11/871,879; 11/862,099; 11/949,490; 12/119,374; 12/141,831; 12/141,864; 12/165,366; 12/843,512; 61/304,148; 61/218,763; 12/723,205; 12/819,959; 61/368,150; 13/026,459; 13/189,330; 13/226,211; 61/606,153; 11/127,055; 11/526,956; 61/291,211; 61/296,391; 12/792,333; 12/793,374; 61/361,208; 61/485,944; 13/175,670; 61/509,994; 61/577,565; and 61/682,117.

### BACKGROUND OF THE INVENTION

The study of the physiology effects of cardiac arrest has been a particular area of interest in recent decades. This is due mainly to cardiac arrest remaining the leading cause of death in the United States. As a result of this focus, a number of approaches to treating cardiac arrest have been developed, which have resulted in significant clinical advances in the field. Despite this progress, greater than 80% of patients who experience sudden and unexpected out of hospital cardiac arrest (OHCA) cannot be successfully resuscitated. The prognosis is particularly grim in patients with a prolonged time between cardiac arrest and the start of cardiopulmonary resuscitation (CPR). A common, harmful, and previously under-recognized contributor to patient morbidity and mortality is reperfusion injury (RI), which typically occurs or is induced after a prolonged period of no blood flow in the OHCA setting.

These persistently high mortality rates have encouraged exploration of new approaches to cardiac arrest with a goal of improving patient outcomes. Despite intensive research over half a century, only small improvements in resuscitation survival outcomes have been observed. Most efforts in the field have focused on improving hemodynamics during CPR. Ideally, any new approaches would complement previously determined methods that improve circulation to the heart and brain after cardiac arrest.

### BRIEF SUMMARY OF THE INVENTION

The embodiments described herein provide devices, systems, and methods for reducing reperfusion injury during performance of CPR as blood flow is returned to tissue following cardiac arrest. According to one aspect, a system for delivering a synthetic surfactant and an anesthetic to an individual to reduce reperfusion injury includes an anesthetic delivery device and a device for administering the synthetic surfactant intravenously or intraosseously. The anesthetic delivery device includes a patient connection mechanism for coupling with an airway of the individual, an intrathoracic pressure regulation (IPR) mechanism that involves changing the pressure in the airway that is coupled with the patient connection mechanism, and an anesthetic delivery mechanism for receiving the anesthetic and for delivering the anesthetic to the individual via the patient connection mechanism.

In some embodiments, the device includes a housing and the anesthetic delivery mechanism includes a chamber for receiving a vial of the anesthetic. In some embodiments, the synthetic surfactant includes poloxamer P188 and the synthetic surfactant is administered within 10 minutes of starting cardiopulmonary resuscitation.

In some embodiments, the device includes a housing having an upper chamber and a lower chamber separated by a filter. In such embodiments, the lower chamber may include an impedance threshold device (ITD) and the upper chamber may include an inlet port for receiving a gas to be delivered to the individual via the patient connection mechanism. The upper chamber may also include an absorbent buffer. In some embodiments, the device further includes an aerosolizer to aerosolize the anesthetic.

According to another aspect, a method of performing cardiopulmonary resuscitation includes administering a synthetic surfactant to an individual receiving cardiopulmonary resuscitation (CPR). The synthetic surfactant may be administered within 10 minutes of starting CPR and may be administered intravenously or intraosseously. The synthetic surfactant may be poloxamer P188. In some embodiments, the synthetic surfactant may be administered as a bolus within 5 minutes of initiating CPR and may be administered to the individual via an IV infusion for a plurality of hours after performing CPR.

In some embodiments, the method may also include administering an anesthetic to the individual. The anesthetic may be administered into the individual's lungs within 30 seconds to 3 minutes after starting CPR. In some embodiments, the anesthetic may be administered prior to performing CPR including chest compressions or defibrillation. In some embodiments, the anesthetic may be administered while performing an enhanced circulation cardiopulmonary resuscitation procedure using one or more or the combination of an impedance threshold device (ITD), a manual or automated active compression decompression (ACD) CPR, and/or an intrathoracic pressure regulator (ITPR) device. In some embodiments, the surfactant and anesthetic may be administered prior to restoration of a heartbeat and/or the anesthetic may be administered to the individual for at least 30 seconds or 5 minutes.

According to another aspect, a method of reducing reperfusion injury after a period of ischemia includes administering a synthetic surfactant to an individual prior to or approximate the time blood supply is returned to tissue of the individual. The method may also include administering an anesthetic concurrently with the synthetic surfactant. The synthetic surfactant may be poloxamer P188 and may be administered intravenously or intraosseously.

According to another aspect, a method may include administering anesthesia and P188 during the performance of cardiopulmonary resuscitation to modulate the autonomic nervous system.

### BRIEF DESCRIPTION OF THE DRAWINGS

The present invention is described in conjunction with the appended figures:

FIG. 1 illustrates a device for administering polymer P188, a nonionic block copolymer surfactant that has rheologic, anti-thrombotic, anti-inflammatory, and cytoprotective activities, and an anesthetic to an individual according to one embodiment of the invention.

FIG. 2 illustrates a device for administering a synthetic surfactant, such as polymer P188, to an individual according to one embodiment of the invention.

FIG. 3 illustrates a system for administering an anesthetic and polymer P188 to an individual according to one embodiment of the invention.

FIGS. 4-6 illustrate various methods according to embodiments of the invention.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE INVENTION

Embodiments of the invention provide devices, systems, and methods for reducing reperfusion injury during performance of CPR as blood flow is returned to tissue following cardiac arrest. During cardiac arrest and immediately after resuscitation, the absence of oxygen and/or other nutrients can create conditions that result in inflammation and/or other damage to tissue and/or cell components. This occurs when the heart stops beating during a cardiac arrest. Blood supply or flow is interrupted to an individual's heart and brain, which may cause damage to the heart and brain tissue, or possibly death of the individual, if left untreated for an extended period of time. Cardiopulmonary resuscitation (CPR) may be performed in order to restart beating of the heart or to establish circulation of blood within the body. CPR typically involves performing chest compressions, either manually or with the assistance of a machine or device; performing artificial respiration to provide oxygen to the body, either manually (e.g., mouth to mouth) or with the assistance of a machine or device; or some combination thereof.

Active compression decompression CPR (ACD CPR) is a type of CPR that has been found beneficial in increasing blood circulation within the body and/or increasing blood oxygenation. ACD CPR increases the amount of blood returned to the heart by enhancing the intrathoracic vacuum or negative pressure during chest wall recoil. The amount of blood circulated within the body during subsequent chest compressions is increased due to the increase in blood that is returned to the heart. Several devices may be used or assist in performing ACD CPR, such as those described in the patents and applications incorporated herein. An Impedance Threshold Device or mechanism (ITD) is a specific device that may be used during performance of CPR or ACD CPR to decrease intrathoracic pressure and improve blood return to the heart. In some embodiments, the ITD device includes a valve that is part of a mask or breathing device (e.g., an endotracheal tube) that opens at a defined high or low pressure. Several ITD devices are described in the patents and applications incorporated herein. A specific ITD device is the ResQPOD® sold by Advanced Circulatory Systems, Inc. Another device, termed the intrathoracic pressure regulator (IPR), enables the users to provide positive pressure ventilation and then generates a low negative pressure within the thoracic to enhance venous blood flow back to the right heart. Embodiments of such devices are described in U.S. patent application Ser. No. 11/679,693, filed Feb. 27, 2007, titled “CPR DEVICES AND METHODS UTILIZING A CONTINUOUS SUPPLY OF RESPIRATORY GASES”, U.S. patent application Ser. No. 13/026,459, filed Feb. 14, 2011, titled “GUIDED ACTIVE COMPRESSION DECOMPRESSION CARDIOPULMONARY RESUSCITATION SYSTEMS AND METHODS”, U.S. patent application Ser. No. 13/189,330, filed Jul. 22, 2011, titled “AIRWAY ADJUNCT RESUSCITATION SYSTEMS AND METHODS”, U.S. patent application Ser. No. 12/119,374, filed May 12, 2008, titled “SYSTEM, METHOD, AND DEVICE TO INCREASE CIRCULATION DURING CPR WITHOUT REQUIRING POSITIVE PRESSURE VENTILATION”, U.S. patent application Ser. No. 12/819,959, filed Jun. 21, 2010, titled “VACUUM AND POSITIVE PRESSURE VENTILATION SYSTEMS AND METHODS FOR INTRATHORACIC PRESSURE REGULATION”, U.S. patent application Ser. No. 13/554,986, filed Jul. 20, 2012, titled “ENHANCED GUIDED ACTIVE COMPRESSION DECOMPRESSION CARDIOPULMONARY RESUSCITATION SYSTEMS AND METHODS”, and U.S. patent application Ser. No. 13/411,230, filed Mar. 2, 2012, titled “CPR VOLUME EXCHANGER VALVE SYSTEM WITH SAFETY FEATURE AND METHODS”, the entire disclosures of which are incorporated herein by reference. This approach also simultaneously lowers intracranial pressure, thereby lowering resistance to forward brain blood flow.

Another form of CPR involves modulating, regulating, or otherwise controlling blood flow to the heart and brain, with or without the administration of a vasodilator drug. This is done so that the vital organs receive blood in a controlled fashion. This may be particularly useful as changes in blood flow may cause the release of endogenous vasodilators. By modulating blood circulation, potential reperfusion injury following CPR may be reduced. The blood flow may be controlled or modulated so that the vital organs slowly receive additional blood over time. Controlling or modulating blood flow in this manner may involve slowly increasing or ramping the amount of blood supplied to the vital organs over time. Similarly, blood may be circulated in a “stutter” fashion where blood is circulated to the vital organs for a certain time, then stopped, then again circulated. Within 2-10 minutes blood flow is then restored to near normal levels and in this manner cells and body organs are protected from the injury associated with reperfusion after cardiac arrest. In some cases, a combination of these methods could be used. This “stutter” CPR approach is more fully described in U.S. Patent Application No. 61/509,994, the complete disclosure of which is herein incorporated by reference.

Other forms of cardiopulmonary resuscitation are also possible. Some are performed manually, others with automated devices. All are designed to improve circulation to the heart and brain. For convenience in describing embodiments of the invention, all forms of cardiopulmonary resuscitation (e.g., ACD CPR, CPR, automated devices, etc.) will be referred to herein as CPR. It should be realized that this description does not limit embodiments of the invention to one particular form of cardiopulmonary resuscitation and that all forms of such resuscitating procedures are contemplated herein. In some instances, the patient's head may be elevated above the heart, such as up to 45 degrees, so that gravity can assist in facilitating venous return from the head to the heart during CPR.

This application is focused on using a polyoxamer, P188 either alone or preferably with an anesthetic to protect the heart and brain against reperfusion injury during and after CPR. Poloxamer 188 (hereinafter P188) is a nonionic block copolymer surfactant that has rheologic, anti-thrombotic, anti-inflammatory, and cytoprotective activities. It has been used in a number of different disease states to treat shock from blood loss and in the setting of ischemia, when not enough blood is circulating to different tissues. A description of a Poloxamer and some of its uses is provided in U.S. Pat. No. 5,605,687, titled “METHODS AND COMPOSITIONS OF A POLYMER (POLOXAMER) FOR REPAIR OF ELECTRICAL INJURY”, the entire disclosure of which is incorporated by reference herein.

In the present application, P188 is described for use in the treatment of patients undergoing CPR, either by itself, or preferably with an anesthetic such as sevoflurane, that also independently confers protection from reperfusion injury after a prolonged period of no blood circulation. In one embodiment, an anesthetic may be delivered to the individual prior to or during reperfusion or blood circulation. For example, an anesthetic may be delivered prior to or during the performance of CPR. In such embodiments, P188 could also be administered intravenously or intraosseously. The anesthetic may be delivered via a device during the performance of artificial respiration while P188 is delivered intravenously. The anesthetic may be delivered with or without an amount of oxygen. The anesthetic may be volatile, or include a volatile agent, so that the anesthetic quickly aerosolizes upon breaking of a vial or other container housing the anesthetic. In other embodiments, the anesthetic and P188 may be delivered concurrently to the individual intravenously, intramuscularly, intraosseously, and the like. The anesthetic may be administered or delivered prior to or simultaneously with performing chest compressions. The polymer, P188, could be administered contemporaneously, via an intravenous or intraosseous route. In another embodiment, the anesthetic and P188 may be administered prior to restoration of blood circulation or reestablishment of a heartbeat. In some embodiments, the anesthetic is administered via an intrathoracic pressure regulation (IPR) device or mechanism. For example, the anesthetic may be fed through an ITD device, such as the ResQPOD®. In yet other embodiments, the P188 may be delivered to an individual as part of a “bundle of care” or CPR cocktail that is given as soon as possible after, or simultaneous with, initiating CPR. In such embodiments, the P188 may be delivered as a bolus. The P188 may optionally also be administered for several hours to the individual via IV infusion. In some embodiments, the P188 and/or anesthetic may be administered and/or the CPR techniques described herein may be performed even when the patient is unable to survive a cardiac arrest. This may be done as a means to maintain the other bodily organs even when the brain is not salvageable, such as for use in transplantation.

In one embodiment, a bundle of care is provided that includes ACD CPR and an ITD or IPR device. CPR, P188, and an anesthetic are delivered in a “stutter-like” manner for the first 2-3 minutes as noted above. The CPR methods and drugs work synergistically to enhance circulation and to deliver the anesthetic and poloxamer to the brain and other key organ. The combined CPR and drug administration optimizes blood circulation to reduce reperfusion injury and the cascade of biochemical processes that can occur when blood is reintroduced into the vital organs after a prolonged period of no blood flow.

Reperfusion injury occurs very early during reintroduction of blood flow during CPR. Applicants have shown that protective strategies to reduce or prevent reperfusion injury improve neurologically sound survival after 15 minutes of untreated cardiac arrest, which physiological advances have never been previously reported. These observations are described in greater detail in U.S. patent application Ser. No. 13/554,986, filed Jul. 20, 2012, titled “ENHANCED GUIDED ACTIVE COMPRESSION DECOMPRESSION CARDIOPULMONARY RESUSCITATION SYSTEMS AND METHODS”, and U.S. patent application Ser. No. 13/554,458, filed Jul. 20, 2012, titled “METHODS AND SYSTEMS FOR REPERFUSION INJURY PROTECTION AFTER CARDIAC ARREST”, the entire disclosures of which are incorporated herein by reference. Pharmacological agents (e.g. cyclosporine A) may provide similar injury protection results to the heart but not the brain.

Intravenous drugs generally have limited applicability during the early phases of CPR due to their dependence on intravenous or intraosseous access, which is generally provided by Advanced Life Support Providers such as medics and physicians. A delay in administration of reperfusion injury protection strategies or agents may result in a loss or signification decrease of benefit when blood flow is introduced or resumed more than three minutes before the agent is administered.

This limitation may be circumvented by several reperfusion injury protection strategies that implement a first reperfusion injury protection strategy at the initiation of CPR and that is followed by the intravenous and/or intraosseous delivery of poloxamers, such as P188 soon thereafter. For example, introduction of controlled pauses during the first 2-3 minutes of CPR provide a significant degree of reperfusion injury protection, as described in U.S. patent application Ser. No. 13/554,458, filed Jul. 20, 2012, titled “METHODS AND SYSTEMS FOR REPERFUSION INJURY PROTECTION AFTER CARDIAC ARREST”, the entire disclosure of which is incorporated by reference herein. Subsequently, as soon as intraosseous and/or intravenous access is obtained, delivery of a poloxamer (e.g., P188) can rapidly provide additional reperfusion injury protection. Similarly, delivery of an anesthetic agent, as described in U.S. Patent Application No. 61/682,117, filed Aug. 10, 2012, titled “SYSTEM AND METHOD FOR ADMINISTERING ANESTHETICS WHILE PERFORMING CPR”, the entire disclosure of which is incorporated by reference herein, at the start of CPR, with or without a controlled pauses strategy as described in part in the '458 U.S. patent application incorporated herein, provides some reperfusion injury protection that can be further augmented by the rapid administration of a poloxamer, such as P188.

In an exemplary embodiment, effective methods of CPR that optimize circulation as soon as CPR is initiated are used. Such methods include the use of an ITD device with conventional manual CPR, use of the combination of ACD CPR and an ITD device, or use of ACD CPR and an intrathoracic pressure regulator. Access to the lungs is virtually immediate through the airways since access to the lungs can be provided with a bag-valve-mask system or asupraglottic airway device and can be implemented within a few seconds of the resuscitation efforts. Inhaled anesthetics such as sevoflurane and isoflurane, helium or xenon can be delivered as a bolus for a short period of time at the first contact with the patients and provide effective protection against ischemia.

Victims of cardiac arrest frequently suffer from lack of blood flow to their entire body (i.e., ischemic injury) and from cellular damage when CPR is started (i.e., reperfusion injury). Both ischemic injury and reperfusion injury are associated with loss of integrity of the cellular membranes, which play a vital role in the function and protection of all cells in the body. This damage is manifest, in part, by an increase in cell membrane and subcellular organelle membrane (e.g., mitochondrial) permeabilization.

It is believed that effective therapy for victims of cardiac arrest should re-establish cell membrane structural integrity, and preferably, should also provide a means for promoting cellular energy store regeneration. It is further believed that an effective therapy may be provided by utilizing a blood-compatible surfactant for sealing vital membranes, especially in the brain and heart after cardiac arrest. The blood-compatible surfactant may also provide protection to most other organs, such as the kidney, liver small intestine, and large intestine, all of which are prone to multi-organ failure unless treated.

In a simplistic model of membrane repair after cardiac arrest and sustained ischemia (lack of blood flow) it could be postulated that upon permeabilization (opening at numerous sites) of the cell membrane, both the hydrophobic region of membrane protein and the hydrophobic moieties, tails of the membrane's lipid bilayer are exposed to the more polar, aqueous environments of the extracellular fluid and the cell cytoplasm. Cytoskeletal elements typically located at or near the internal layer of the cell membrane might be expected to react in such a way as to form at least a partial barrier to the escape of intracellular organelles and compartments. However, ions would be expected to both escape from and enter into the cell down their concentration gradients, and other small molecules to pass through the many small holes that result in the permeabilization of the membrane. If the extent of the damage to the cell membrane is severe, it could be possible that a significant amount of cytoplasm, including macromolecules and small organelles, may leak out of the cell.

In the setting of a prolonged ischemic injury, the lipid molecules in the membrane bilayer nearest the openings would be expected to respond to exposure to the polar environment by reorienting themselves such that their hydrophilic head groups would turn toward the polar solution at the point of exposure. This would result in the isolation of the hydrophobic tails of the lipid molecules within the membrane from the aqueous intra- and extracellular space. Membrane fusion and repair of such patencies would occur as the newly formed hydrophilic edges of the patencies move into apposition with each other. Methods that include a way to restoring the permeabilized cells' integrity, such as those described herein, provide an important advance in the treatment of injuries involving cell membrane permeabilization.

Surface active copolymers, also termed block polymer nonionic surfactants, are surface active agents synthesized by the sequential addition of two or more alkylene oxides to a low molecular weight water soluble organic compound containing one or more active hydrogen atoms. Exemplary groups of surface active copolymers include: poloxamers, meroxapols, poloxamines, and PLURADOT® with respect to the polymer's synthesis. In the last group of proposed copolymers, the alkaline catalyst may be neutralized and removed from the final product.

As described in the '687 U.S. patent application incorporated herein, poloxamers are synthesized by sequential addition of propylene oxide, followed by ethylene oxide, to propylene glycol. In the case of the poloxamers this constitutes the water-soluble organic component of the polymer. By contrast the inner polyoxy-propylene glycol is the hydrophobic portion of the poloxamer. This group changes from a water-soluble to a water-insoluble polymer with molecular weights above 750 Daltons. Adding ethylene oxide in the final step makes the molecule water-soluble.

In some exemplary embodiments, a poloxamer with a molecular weight of between 2,000 and 20,000 Daltons is used. A poloxamer within this molecular weight range remains soluble in water while minimizing or eliminating any potential toxicity. In some embodiments, the poloxamer's hydrophobic group has a molecular weight range from approximately 950-4,000 Daltons. In such embodiments, the hydrophilic groups may constitute approximately 45-95% by weight of the poloxamer. In an exemplary embodiment, the hydrophobic group has a molecular weight of 1,750-3,500 Daltons and the hydrophilic groups constitute between 50-90% by weight of the molecule. The relative amounts of hydrophile and the molecular weight of the hydrophobe are typically important to several of the poloxamer's properties, such as its solubility in water and its interactions with hydrophobic groups. The above described ranges provide a maximum effectiveness while minimizing or eliminating toxicity.

The method of synthesis of the meroxapol series, the poloxamines, and PLURADOT® trifunctional alcohol, such as glycerine or trimethylpropane, are further described in the '687 U.S. patent application, which is incorporated herein. All four nonionic polymers are alike in that they derive their solubility in water from hydrogen bond formation between the many oxygen atoms on the copolymer and protons in the water. With a rise in temperature of a solution containing a nonionic surfactant, the hydrogen bonds are typically broken and the copolymer clouds out of solution. When performing CPR and/or using advanced resuscitation tools, such as the ACD CPR device and the ITD described herein, poloxamers are surprisingly capable of restoring cardiac and brain function after prolonged cardiac arrest.

In a specific embodiment, the synthetic surfactant, poloxamer 188 (P188), has been determined to provide significant reperfusion injury protection after prolonged untreated cardiac arrest secondary to ventricular fibrillation. It has been further determined that the use of P188, when administered concurrently with the combination of ACD CPR, an ITD, controlled pauses (i.e., stuttering), and/or sevoflurance as described in the '117 U.S. Provisional patent incorporated herein, provides additional significant neuro and cardiac protection as manifest by the restoration of normal cardiac and neurological function in greater than 50% of animals within 48 hours after resuscitation as well as restoration of normal serum markers for the measurement of cardiac, liver, and kidney function. These results are in contrast to the popular belief that the brain dies after 4-6 minutes of no blood flow.

In one embodiment, P188 may be administered intravenously along with the performance of ACD CPR, an ITD, controlled pauses (i.e., stuttering), and/or the administration of sevoflurane. An exemplary embodiment includes administering the P188 and/or sevoflurane while performing each of the above processes. Other embodiments may include co-administering a high energy phosphate compound, such as, for example, 1% w/v ATP and 1% w/v MgCl with the poloxamer and/or anesthetic. This may allow for concomitant re-establishment of the cellular energy charge along with repair of the cell membrane, thereby enhancing cell survival following the prolonged ischemic injury. In another embodiment, the administration could include approximately 10% w/v phosphocreatine, a phosphoric acid derivative of creatine which contains an energy-rich phosphate bond, in place of the ATP-MgCl as the high energy phosphate compound. In yet other embodiments, the P188 may be administered intraosseously, ionophoreticly (e.g., through the skin), and the like.

Given the numerous harmful effects that are associated with lack of adequate blood supply and nutrients (including oxygen when a cell, organ, or whole body is deprived of blood circulation), one advantage of embodiments described herein is in reducing, minimizing, and/or eliminating the potential harm associated with both ischemic injury (lack of blood flow) and reperfusion injury (harm due to the rapid reintroduction of blood flow organs that have not been perfused with blood for a period of time). The embodiments described herein provide protection at multiple different levels in a cellular and intracellular bundle of care approach to this otherwise lethal condition. The invention helps to protect the cell membrane with the delivery of synthetic surfactants, like P188, which plugs the holes in the cell membranes that are formed as a result of the ischemic injury. The poloaxamers and like compounds bind to the cell membranes and markedly reduce permeability that has been induced by the ischemic injury. The embodiments described herein also deliver blood flow to the organs and cells more effectively, but in a way that reduces reperfusion injury. Blood delivery strategies such as “stutter” CPR as well as the anesthetics reduce the intracellular injury that is attributable to mitochondrial dysfunction and metabolic and enzymatic abnormalities associated with both poor perfusion and reperfusion injury. Thus, synthetic surfactants reduce cell membrane injury and the anesthetics, and similar compounds such as inert gases, beneficially affect the internal cell functionality and metabolic state.

The embodiments also provide a non-invasive way to deliver these therapies to the organs and cells and subcellular elements using circulatory enhancement devices that have been shown to provide normal blood flow to the brain. Accordingly, this biophysical and biochemical bundle of care for cardiac arrest may include four elements: 1) non-invasive CPR devices that provide optimal flow and circulation to the heart and brain, 2) enhanced CPR that reduces reperfusion injury (e.g., “stutter” CPR), 3) synthetic surfactants that reduce cell permeability resulting from cardiac arrest and ischemia, and 4) anesthetics and similar compounds that beneficially affect intracellular metabolism and mitochondrial function.

In regards to delivering an anesthetic, in one embodiment, the anesthetic is administered as an enhanced circulation procedure is performed, such as ACD CPR or CPR using an ITD or IPR device. The enhanced circulation may deliver the anesthetic to the brain more quickly when compared to conventional CPR or other circulation procedures. Enhanced delivery time of the anesthetic to the brain and/or other organs may aid in reperfusion injury protection by allowing the anesthetic to quickly anesthetize the brain and/or other organs. Tissue damage due to reperfusion injury may be reduced when the anesthetic is delivered to the brain quickly after restoration of blood flow or circulation, such as after beginning chest compressions. In one embodiment, the anesthetic is delivered to the brain within the first several minutes by performing enhanced circulation procedures (e.g., ACD CPR or CPR using an ITD device). In a specific embodiment, the anesthetic is delivered to the brain within the first two minutes or within the first minute after reestablishing blood flow or circulation.

In one embodiment, the anesthetic is administered to the individual in a single dose, although in other embodiments the anesthetic may be delivered in several doses. For example, a single dosage may be administered via intravenous, intramuscular, or intraosseous injection, or may be administered via inhalation. The anesthetic may be volatilized and delivered via one or more respirations. In inhalation administration procedures, the anesthetic may be delivered for 30 seconds, 2 minutes, and more commonly for 5 minutes or more. In another embodiment, a dosage of the anesthetic is delivered during each respiration or repeated respirations for a portion or the entire period of time an individual is receiving artificial respiration or ventilation. In such embodiments, delivery of the anesthetic may last for 30 minutes or more, often at reduced dosage amounts. In one embodiment, the anesthetic, such as sevoflurane, may be delivered in a dosage amount of 2-4 vol % over one or more respirations. In another embodiment, the anesthetic may be delivered during the short periodic pauses in a “stutter” CPR process.

In one embodiment, the administered anesthetic includes sevoflurane. Other volatile anesthetics that may be administered include: halothane, enflurane, and desflurane isoflurane, xenon, helium and the like. Sevoflurane has shown a significant advantage over other inhaled anesthetics providing significant protection from reperfusion injury. Sevoflurane also has less of cardiodepressant effects. These anesthetics may provide protective effects when administered before or during CPR and help stabilize the brain, heart, or body to help the heart, brain, and/or body to stay fresh. It is believed that some anesthetic agents provide these effects by attenuating the mitochondrial membrane permeability, or manipulating and protecting the mitochondrial membrane pores. It is believed that during ischemia, permeability pores in the mitochondria cell membrane open up causing the mitochondria to swell. In such a condition, the mitochondria are destroyed or damaged when blood flow is reestablished. The anesthetic may help protects against such damage, especially if the anesthetic is delivered or delivery is initiated early in reestablishing blood flow. Some volatile anesthetics like Xenon provide anesthesia by non-competitive inhibit N-methyl-D-aspartate receptors. Due to the fact that reperfusion injury occurs very early during the resuscitation efforts (within 2-3 minutes from the imitation of CPR) it is preferable that the anesthetic is delivered or delivery is initiated within the first 5 minutes of CPR efforts. In other embodiments, when it is feasible and for optimal protection, the anesthetic is delivered or delivery is initiated within the first 2-3 minutes of CPR. Early delivery of the anesthetic reduces or prevents the initial shock the organs of the body experience by the first pass of blood and oxygen in the blood and/or prevent or reduce the effects of adverse toxins that may accumulate in the blood during ischemia. Delivery of the anesthetic may also aid in resuscitating the individual. For example, in animal studies involving administration of an anesthetic gas during CPR, the animal was resuscitated after only one or two shocks. Further, within an hour or two of resuscitation, most or all the animals' functions were normal even without administration of other drugs, such as inotropic drugs.

It is believed that the anesthetic may also help modulate the autonomic nervous system by altering the body's response to adrenaline and/or the nervous system's response to restarting blood flow or circulation. Subsequent to establishing blood circulation and/or reestablishing a heartbeat, the body experiences a surge of adrenaline and other stress hormones. The anesthetic may modulates the body's response and/or blunt the nervous system's response to reduce the adrenaline and/or other stress hormones levels associated with reestablished circulation.

The anesthetic may further provide protection by allowing or enabling energy stores of neurons to be replenished before they can be activated again. This may help to reduce the mismatch between energy supplies and energy stores in the brain and/or other areas of the body. For example, the anesthetic may reduce the energy need of the brain thereby ensuring that the brain does not exhaust its fuel quickly, thus extending the functional time of the brain. Administering and circulating the anesthesia to the brain quickly (i.e., early in the CPR process) may stabilize the brain cells metabolically, and preserve them so that they remain subdued. Similarly, the anesthetic may reduce the activity of the central nervous system thereby reducing its need for fuel. The anesthetic may prevent or prolong these systems from functioning in an energy deprived state or condition.

In one embodiment, the anesthetic is delivered prior to establishment of a heartbeat or blood circulation, artificial or otherwise (e.g., via chest compressions, defibrillation, and the like). More commonly, the anesthetic is delivered simultaneously with or quickly after establishing a heartbeat or blood circulation. Preferably, the anesthetic is delivered within the first 5 minutes of establishing a heartbeat or blood circulation, within the first 3 minutes, or within the first 2 minutes.

In animal studies where an anesthetic gas was administered as described herein, heart muscle damage was drastically reduced. In many of the animals that received the anesthetic, the heart was beating normally and blood pressure was normal approximately one hour after resuscitation. The ability to resuscitate the animal was also enhanced.

These and other aspects of the invention will become more evident with reference to the figures described below.

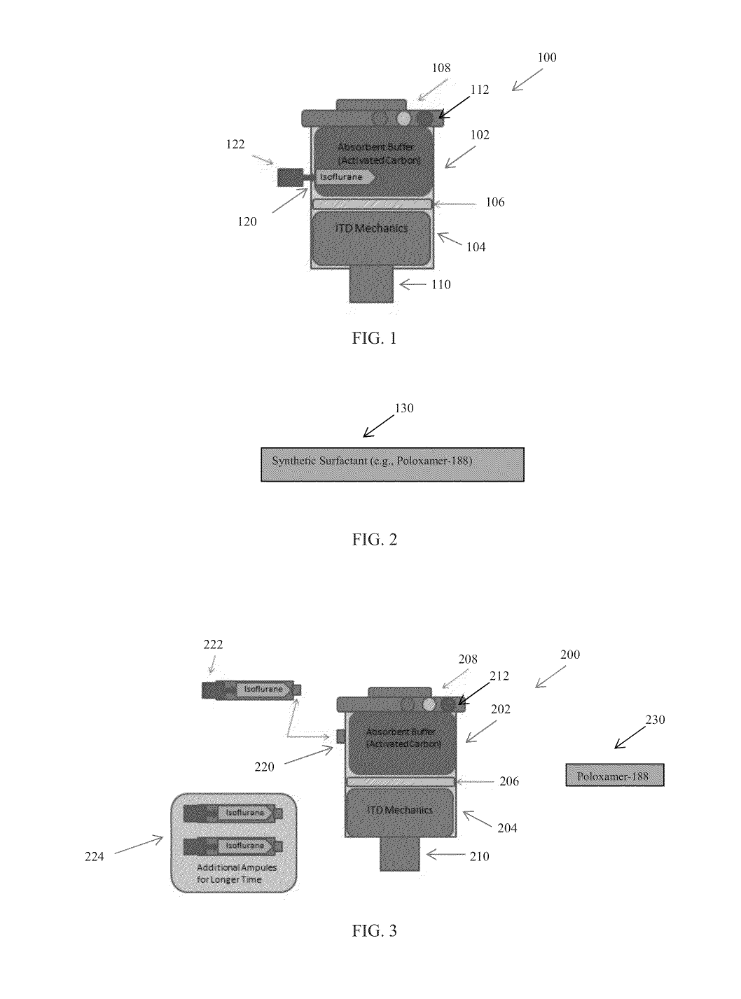

FIG. 1 illustrates a device 100 or system for administering an anesthetic to an individual to reduce reperfusion injury. Device 100 may be used while performing CPR. Device 100 includes a housing having an inlet or ventilation port 108 and an outlet or patient port 110. Inlet port 108 is configured to connect with a ventilation source or device that is used to provide oxygen to the individual, such as a compressible bag, a mechanical device, an anesthesia machine, a ventilator, and the like. Inlet port 108 may be pivotally coupled with the housing to allow the ventilation source or device to be attached and used within a wide range of angle. The housing may also include various electronics or guidance systems 112 that guide a user or operator in performing CPR. For example, guidance system 112 may guide a user on proper ventilation rate and respiration duration and/or may indicate a timing of when to provide subsequent respirations. One or more lights may be used that indicate when a breath or respiration should be provided, the duration of the respirations that should be administered, when chest compressions should be performed, and the like. In one embodiment, the lights are configured to indicate respirations and chest compressions according to the “stutter” CPR process so that CPR process involved short periodic pauses. The anesthetic may be administered during one or more of these short periodic pauses. Outlet port 110 typically connects with tubing and/or a mask that is placed over the patient to deliver oxygen and/or inhalants to the patient, such as an inhaled anesthetic.

Device 100 may also include an upper chamber 102 and a lower chamber 104 separated by a filter 106 material or membrane. Upper chamber 102 may include a port 120 or chamber for receiving the anesthetic. For example, in one embodiment, an ampule or vial of the anesthetic may be inserted within port 102. A fracture button 122 may be then be depressed to break the ampule or vial and thereby release the anesthetic within upper chamber 102. The fracture button 122 may also be used to inject the anesthetic within upper chamber 102. As described herein, the anesthetic may include a volatile agent that allows the anesthetic to quickly aerosolize. Upper chamber 102 may include an absorbent buffer, such as activated carbon, which is used as a capture chamber to scavenge the gas and keep the anesthetic from escaping. Using the scavenging system, the gas could be recirculated. In one embodiment, the activated carbon includes charcoal.

The vial or ampule inserted or injected within port 102 may include a single dose of the anesthetic. In one embodiment, the single dose provides approximately 30 seconds worth of inhalant anesthetic. In another embodiment, the single dose provides approximately 5 minutes or more worth of the inhalant anesthetic. In yet another embodiment, the single dose provides between about 30 seconds and 5 minutes worth of inhalant anesthetic. In other embodiments, multiple vials/doses may be administered to the patient depending on the volume of anesthetic gas, respiration duration, and/or frequency of respirations desired. In one embodiment, approximately a half a liter of anesthetic gas is provided in a single respiration, which may be administered in about 30 seconds. In another embodiment, a series of about 5 respirations are provided where each respiration contains about half a liter (i.e., roughly 500-525 cubic centimeters) of anesthetic gas so that a total volume of roughly 2500 cubic centimeters of anesthetic gas is administered. Administration of the anesthetic gas may be initiated within the first 5 minutes of performing CPR, and preferably within the first 3 minutes or 2 minutes. Preferably, the anesthetic is administered within the first 2-3 minutes or less of starting CPR and is administered for at least 30 seconds and more commonly about 5 minutes or more. In another embodiment, the anesthetic may be delivered during the short periodic pauses in a “stutter” CPR process. The above described volumes and durations should be sufficient to ensure that the brain and/or other organs are sufficiently anesthetized early in CPR process, which may effectively protect against reperfusion injury by reducing or eliminating damage to these organs and tissue.

As described herein, delivering the anesthetic gas early in the reperfusion process may be important to protecting against reperfusion injury because it is believed that reperfusion injury often occurs or begins within the first 3 minutes of reestablishing blood flow. Although the anesthesia is typically administered early in the process, in one embodiment the anesthetic gas is delivered during a majority or the entire CPR process or subsequently delivered up to 24 hours while the patient is recovering.

In one embodiment, a liquid anesthetic is delivered or injected into upper chamber 102 and subsequently aerosolized via an aerosolizer. The liquid anesthetic may be aerosolized to provide the above described amount of inhalant anesthetic. Also, although shown as being positioned in the upper chamber 102, the vial or ampule chamber 120 may be positioned in the lower chamber 104 in some embodiments.

Filter 106 is used to filter gas that passes from upper chamber 102 through lower chamber 104 and to the patient or individual. Specifically, filter 106 is used to remove bacteria and other microscopic particles that could infect or otherwise harm the patient or individual. In one embodiment, filter 106 removes bacteria as small as 10 microns. The aerosolized anesthetic gas passes through filter 106 along with the gas from inlet port 108 and into lower chamber 104, which is positioned below filter 106. In one embodiment, the gas from inlet port 108 is oxygen that is mixed with the anesthetic in upper chamber 102. Lower chamber 104 includes an intrathoracic pressure regulation (IPR) mechanism or a device to prevent or impede respiratory gases from flowing to the lungs, or to otherwise regulate flow through or pressure within the patient's airway. In one embodiment, lower chamber 104 includes an impedance threshold device (ITD), such as an inspiratory limb flow control assembly or inspiratory valve mechanism. As described above, the ITD or IPR device functions to increase blood return to the heart thereby improving blood flow during subsequent chest compressions. Exemplary ITDs that may be used with device 100 include those described in U.S. Pat. Nos. 5,551,420; 5,692,498; 5,730,122; 6,062,219; 6,155,257; 6,224,562; 6,234,916; 6,526,973; 6,604,523; 6,776,156; 6,986,349; 7,195,012; and 7,204,251; and U.S. Provisional Patent Application No. 61/577,565, the complete disclosures of which are herein incorporated by reference.

In some embodiments, an anesthetic may be delivered concurrently with a surfactant, such as poloxamer P188. The synthetic surfactant may be used to provide significant neuro and/or cardiac protection. The synthetic surfactant may be delivered via a device 130 like that illustrated in FIG. 2. In some embodiments, the P188 may be delivered intravenously, intramuscularly, intraosseously, and the like, in which case the device 130 typically includes a needle and/or syringe that is designed to penetrate tissue in the appropriate manner. In some embodiments, the P188 may be administered to a patient during CPR by itself via device 130. Stated differently, the surfactant may be administered to a patient during a CPR procedure without administering an anesthetic. The surfactant is typically delivered to the patient via device 130 within the first 10 minutes of starting CPR, and preferably within the first 5 minutes, 3 minutes, or 2 minutes, so as to quickly provide neuro and/or cardiac protection. In one embodiment, device 130 is used to deliver the surfactant intravenously during the performance of artificial respiration and/or during chest compressions. The surfactant used (e.g., P188) may have the molecular properties described above and/or be delivered in the amounts described herein.

FIG. 3 illustrates another embodiment of a system for administering an anesthetic and surfactant (e.g., P188) to an individual to reduce reperfusion injury. The system includes a device 200 that is similar to device 100 in that it includes a patient port 210, a ventilation port 208, various electronics or guidance systems 212, an upper chamber 202, a lower chamber 204, a filter 206 separating the upper chamber and lower chamber, and an anesthetic port 220 within which a liquid anesthetic 222 may be injected. Anesthetic port 220 may an aperture or plug that extends from the housing and that couples with a distal end of the vial or ampule containing the liquid anesthetic 222. The vial or ampule containing the liquid anesthetic 222 could be broken or cracked open and injected into upper chamber 202, or in some embodiments, lower chamber 204. In some embodiments, the liquid anesthetic may be volatile or include a volatile agent so that it instantly forms into a gas upon injection. In other embodiments, upper chamber 202 includes an aerosolizer that aerosolizes the liquid anesthesia. According to one embodiment, the vial or ampule of liquid anesthesia may be administered to the patient over a 30 second interval, 2 minute interval, 3 minute interval, 5 minute interval, and the like. According to another embodiment, additional vials or ampules 224 of the anesthesia may be used depending on the amount of anesthesia to be administered and the duration that the anesthesia is to be administered. The system further includes a device 230 that is used to administer the surfactant. Device 230 typically includes a needle and/or syringe that is configured to deliver the surfactant intravenously, intramuscularly, intraosseously, and the like. The surfactant is delivered concurrently with the anesthetic during the performance of CPR and may be delivered in the amounts described herein and/or have the molecular properties described herein.

According to one embodiment, a method of using the device 100 or 200 includes a rescuer or user of the device 100 or 200 arriving at a location where CPR is needed. The rescuer opens a vial or ampule of the anesthetic gas and then inserts the anesthetic gas or injects the gas into device 100 or 200 via respective chambers 120 or 220. The rescuer then begins to ventilate the individual needing CPR with the anesthetic gas, plus/minus any oxygen as desired. The rescuer may also concurrently deliver an effective amount of surfactant (e.g., P188) to the individual via device 130 or 230 as needed. Ventilation occurs for 30 seconds, 2 minutes, 3 minutes, 5 minutes, or more depending on the individual's need and or other factors. Additional vials or ampules may be needed to provide the desired ventilation. The rescuer then performs or simultaneously performs chest compressions or defibrillation to establish blood circulation within the individual's body. The individual could then be ventilated with or without the anesthetic gas.

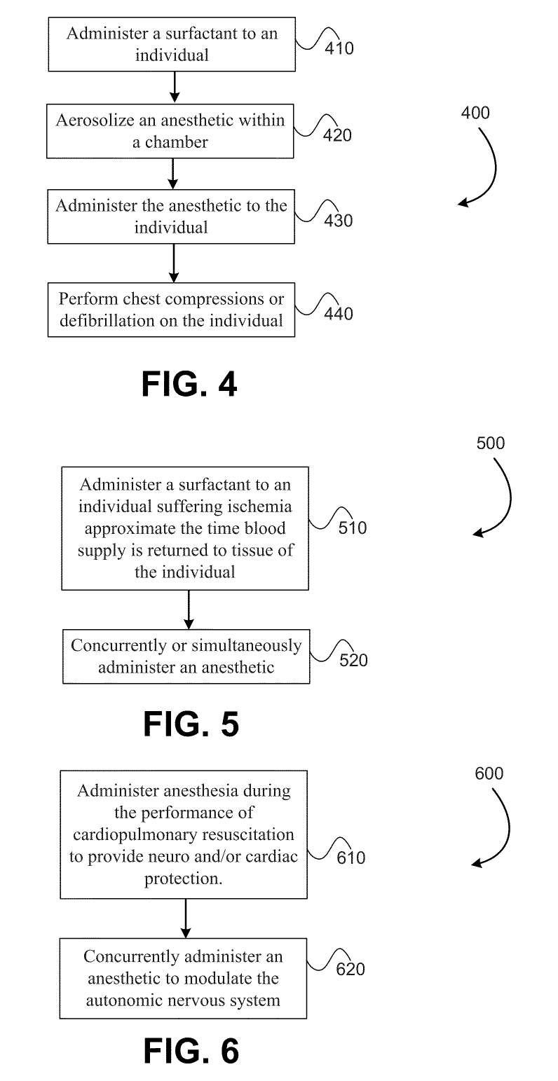

FIG. 4 illustrates a method 400 of performing cardiopulmonary resuscitation on an individual while administering a surfactant and/or an anesthetic to the individual. At block 410, a surfactant, such as P188 and the like, is administered to an individual via an administration device, such as a needle and syringe. The surfactant may be administered intravenously, intramuscularly, intraosseously, as a bolus, and the like. The surfactant may be delivered in an amount described herein and/or have the molecular properties described herein. The surfactant may provide neuro and/or cardiac protection to an individual suffering cardiac arrest and/or another condition. In some embodiments, the surfactant may be delivered to an individual as part of a “bundle of care” or CPR cocktail that is given as soon as possible after, or simultaneous with, initiating CPR. In such embodiments, the surfactant may be delivered as a bolus. The surfactant may optionally also be administered for several hours to the individual via IV infusion. In some embodiments, the surfactant and/or an anesthetic may be administered and/or the CPR techniques described herein may be performed even when the patient is unable to survive a cardiac arrest. This may be done as a means to maintain the other bodily organs even when the brain is not salvageable, such as for use in transplantation.

Blocks 420 and 430 represent administration of an anesthetic and need not be performed when the surfactant is administered on its own. At block 420, an anesthetic is aerosolized within a chamber, such as by injecting anesthesia into the chamber from a vial or ampule. The anesthesia may aerosolize on its own or be aerosolized with the use of a device. As described above, in some embodiments the anesthetic may be administered intravenously, intramuscularly, intraosseously, and the like instead of using an aerosolizer. In such embodiments, blocks 420 and 430 are not performed. At block 430, the anesthesia is administered to the individual, such as by providing artificial respiration to the individual via a ventilator, a ventilation bag, and the like. Artificial respiration may be provided to the individual in accordance with known CPR techniques or procedures. In one embodiment, the anesthetic is administered to the individual for at least 30 seconds. In another embodiment, the anesthetic is administered to the individual for at least 5 minutes. As described above, the anesthetic may be administered for more or less time than this depending on the individual's needs and/or other circumstances. The CPR procedure may utilize an IPR device, such as an ITD, through which the anesthetic may flow.

According to one embodiment, the anesthesia may be mixed and administered with oxygen. In another embodiment, a series of anesthesia treatments may be administered with gaps between subsequent treatments. For example, artificial respiration may be provided at repeated intervals after chest compressions or defibrillation is administered according to known guidelines or procedures (e.g., 2 breaths for every 30 compressions). Anesthesia may be administered during an initial or first set of artificial respirations, but not administered during the next or second set of artificial respirations. The anesthesia may then again be administered during a third set of artificial respirations, but not administered during a fourth set of artificial respirations. This process may continue for as long as artificial respiration is needed. In another embodiment, the anesthesia is administered every tenth artificial respiration so that a gap of approximately nine respirations occurs between subsequent anesthesia administrations. It should be realized that other anesthesia administration/gap combinations are possible depending on what is deemed appropriate.

In one embodiment, oxygen is administered during artificial respiration when the anesthesia is not administered. In another embodiment, oxygen is administered during each or most of the artificial respirations and, when administered, the anesthesia is mixed and administered with the oxygen. In another embodiment, the oxygen or gas administered is recirculated.

At block 430, chest compressions or defibrillation is provided to the individual in accordance with known CPR techniques or procedures. In one embodiment, the surfactant and/or anesthetic is administered to the individual prior to performing any chest compressions or defibrillation so that the surfactant and/or anesthesia is delivered to the body, or portions thereof, prior to or simultaneously with reestablishing blood circulation. In another embodiment, the surfactant and/or anesthetic is administered to the individual simultaneously with performing any chest compressions or defibrillation or after one or more chest compressions or defibrillation procedures have been performed. In either embodiment, the surfactant and/or anesthetic may be administered early in the CPR process and prior to restoration of a heartbeat. According to another embodiment, the surfactant and/or anesthetic is administered while performing enhanced circulation cardiopulmonary resuscitation, such as ACD CPR or CPR using an ITD or IPR device.

FIG. 5 illustrates a method 500 of reducing reperfusion injury after a period of ischemia. At block 510, a surfactant (e.g., P188) is administered to an individual suffering ischemia prior to or approximate the time blood supply is returned to tissue of the individual. At block 520, an anesthetic is concurrently or simultaneously administered with the surfactant to the individual. The surfactant and/or anesthetic may be administered during the administration of cardiopulmonary resuscitation to the individual.

FIG. 6 illustrates another method 600. At block 610, a surfactant is administered during the performance of cardiopulmonary resuscitation to provide neuro and/or cardiac protection to an individual suffering cardiac arrest and/or another condition. At block 620, an anesthetic is concurrently administered to modulate the autonomic nervous system.

### Experimental

The above described process was conducted in pigs in which cardiac arrest was induced. Cardiac arrest was induced by generating ventricular fibrillation (VF) and pigs were left in untreated cardiac arrest for 17 minutes. CPR was then initiated with an ACD CPR device with an ITD and inhaled sevoflurane anesthetic (i.e., a 3% concentration) was introduced via an anesthesia machine. After 30 seconds of CPR, compressions were stopped and two positive pressure breaths were delivered from the anesthesia machine containing the sevoflurane. Twenty seconds later CPR was resumed without positive pressure breaths for 20 seconds and then compression were intentionally stopped and 2 positive pressure breaths were again delivered as described immediately above. This sequence was repeated for a total of 3 pauses. Then continuous ACD and ITD CPR was provided for a total of one minute without interruption. It should be realized that the above process illustrates merely a single approach and that variations of this approach are possible (e.g., more pauses, slightly shorter or longer pauses, and the like).

A defibrillator shock was then delivered, with the option of providing some adrenalin in the range of 0.05-1.0 mg/70 kg person, during the minute of continuous chest compressions. Following successful defibrillation, the animals were cooled by surface cooling and administration of 11 of ice cold saline. They were weaned from the mechanical ventilator and placed back in their pens following extubation.

As described herein, a simple mobile anesthesia device that can deliver a bolus of inhaled anesthetic of known concentration for the first 3-5 minutes of CPR via either an endotracheal tube, facemask, a supraglottic device, and the like, is feasible.

It should be appreciated by those of skill in the art that the techniques and devices disclosed above represent embodiments which may be used in practicing the invention. However, those of skill in the art should, in light of the present disclosure, appreciate that many changes can be made in the specific embodiments and obtain a like or similar result without departing from the spirit and scope of the invention. It should be further appreciated that the embodiments described herein, which in some instances highlight a bundled approach to preserving the biophysical and biochemical properties of cells and organs exposed to prolonged periods of no or low blood flow, may be of particular benefit in the treatment of not only cardiac arrest but patients suffering from a heart attack or a stroke. Further, this approach, when taken in cardiac arrest patients, may result in better organ preservation even if the patient remains comatose. In such instances, the organs may thus be healthier and better suited for transplantation.

What is claimed is:

1. A system for delivering a synthetic surfactant and an anesthetic to an individual to reduce reperfusion injury, the system comprising:
an anesthetic delivery device comprising:
a patient connection mechanism for coupling with an airway of the individual;
an intrathoracic pressure regulation (IPR) mechanism that involves changing the pressure in the airway that is coupled with the patient connection mechanism; and
an anesthetic delivery mechanism for receiving the anesthetic and for delivering the anesthetic to the individual via the patient connection mechanism; and
a device for administering the synthetic surfactant intravenously or intraosseously.

2. The system of claim 1, wherein the device comprises a housing, and wherein the anesthetic delivery mechanism comprises a chamber for receiving a vial of the anesthetic.

3. The system of claim 1, wherein the synthetic surfactant comprises poloxamer P188, and wherein the synthetic surfactant is administered within 10 minutes of starting cardiopulmonary resuscitation.

4. The system of claim 1, wherein the device comprises a housing having an upper chamber and a lower chamber separated by a filter, wherein the lower chamber comprises an impedance threshold device (ITD), and wherein the upper chamber comprises an inlet port for receiving a gas to be delivered to the individual via the patient connection mechanism.

5. The system of claim 4, wherein the upper chamber further comprises an absorbent buffer.

6. The system of claim 1, wherein the device further comprises an aerosolizer to aerosolize the anesthetic.

7. A method of performing cardiopulmonary resuscitation comprising:
administering a synthetic surfactant to an individual receiving cardiopulmonary resuscitation (CPR).

8. The method of claim 7, wherein the synthetic surfactant is administered within 10 minutes of starting CPR, and wherein the synthetic surfactant is administered intravenously or intraosseously.

9. The method of claim 8, wherein the synthetic surfactant comprises poloxamer P188.

10. The method of claim 7, further comprising administering an anesthetic to the individual.

11. The method of claim 10, wherein the anesthetic is administered into the individual's lungs within 30 seconds to 3 minutes after starting CPR.

12. The method of claim 10, wherein the anesthetic is administered prior to performing CPR including chest compressions or defibrillation.

13. The method of claim 10, wherein the anesthetic is administered while performing an enhanced circulation cardiopulmonary resuscitation procedure using one or more or the combination of an impedance threshold device (ITD), a manual or automated active compression decompression (ACD) CPR, and an intrathoracic pressure regulator (ITPR) device.

14. The method of claim 10, wherein the surfactant and anesthetic are administered prior to restoration of a heartbeat.

15. The method of claim 10, wherein the anesthetic is administered to the individual for at least 30 seconds or 5 minutes.

16. The method of claim 7, wherein the synthetic surfactant is administered as a bolus within 5 minutes of initiating CPR, and wherein the synthetic surfactant is administered to the individual via an IV infusion for a plurality of hours after performing CPR.

17. A method of reducing reperfusion injury after a period of ischemia, the method comprising:
administering a synthetic surfactant to an individual prior to or approximate the time blood supply is returned to tissue of the individual.

18. The method of claim 17, further comprising administering an anesthetic concurrently with the synthetic surfactant.

19. The method of claim 18, wherein the synthetic surfactant comprises poloxamer P188, and wherein the synthetic surfactant is administered intravenously or intraosseously.

20. A method comprising:
administering anesthesia and P188 during the performance of cardiopulmonary resuscitation to modulate the autonomic nervous system.
